(19) United States
(12) Patent Application Publication
(10) Pub. No.: US 2025/0221693 A1
(43) Pub. Date: Jul. 10, 2025

(54) Steerable Articulating Surgical Instrument

(71) Applicant: Ihsan Tasci, Cankaya-Ankara (TR)

(72) Inventor: Ihsan Tasci, Cankaya-Ankara (TR)

(21) Appl. No.: 18/406,165

(22) Filed: Jan. 7, 2024

(51) Int. Cl. A61B 17/00; A61B 17/28

(52) U.S. Cl. CPC A61B 17/00234; A61B 17/28; A61B 2017/00314; A61B 2017/00323; A61B 2017/00367; A61B 2017/00862; A61B 2017/00982

(57) ABSTRACT

Present invention provides a steerable articulating surgical instrument comprising an external hollow shaft, wherein the external hollow shaft comprises a bendable portion and a steering arrangement adapted to apply a bending force to the bendable portion, wherein the bendable portion comprises a resilient bendable member adapted to resist the bending force.

### TECHNICAL FIELD OF THE INVENTION

Present invention relates to a steerable articulating surgical instrument especially for minimally invasive procedures.

### BACKGROUND OF THE INVENTION

In endoscopic surgery and especially in laparoscopic surgery, steerable articulating surgical instruments such as forceps, graspers or needle holders are used. Such instruments have a longitudinal shaft with an operational end effector to operate at a site of surgery. The end effector may reach the site of surgery by bending, rotating and articulating the longitudinal shaft. The steerable articulating surgical instruments comprise moving mechanisms including universal joints, scissor mechanisms and telescopic rotating and extending shafts to transmit steering control instructions of an operator to the longitudinal shaft via a handle to bend, rotate and articulate accordingly. The number and arrangement of the moving mechanisms affect the motion capability and stability of the end effector and the overall size of the instrument. Especially, the movement capability of a needle holder is limited by the rotation capability of the needle holder around its own axis and flexibility of the longitudinal shaft. Further, strong forces may be applicable to the flexible shaft in three dimensions. Further, the movements of the end effector have to be smooth and strong.

### BRIEF DESCRIPTION OF THE PRESENT INVENTION

Present invention provides a steerable articulating surgical instrument with improved motion transmission capabilities and stability while maintaining a compact structure.

Present invention provides a steerable articulating surgical instrument, comprising:
an external hollow shaft extending between a proximal end and a distal end opposite to the proximal end, wherein the external hollow shaft comprises a bendable portion adapted to allow deflection of the distal end to a deflected state,
a steering arrangement adapted to apply a bending force to the bendable portion, wherein the bendable portion comprises a resilient bendable member adapted to resist the bending force. The resilient bendable member is adapted to apply retraction of the deflected distal end to an undeflected state.

The resilient bendable member is adapted to store mechanical energy during bending of the bendable portion when the bending force is applied and to release stored mechanical energy when the bending force is removed. The stored mechanical energy is released to retract the deflected distal end to an undeflected state.

The external hollow shaft extends along a central longitudinal axis, wherein the bending force is applied such that the bendable portion and hence the distal end is deflected from the central longitudinal axis to move radially away from the central longitudinal axis.

The steering arrangement is adapted to apply the bending force to urge the bendable portion to a bent state, wherein the distal end is deflected.

The resilient bendable member is adapted to apply retraction such that to align the bendable portion along the central longitudinal axis and to support the bendable portion to return to an unbent state, wherein the distal end is not deflected.

The resilient bendable member is made of an elastic and rigid material, preferably steel, more preferably spring steel. The resilient bendable member may comprise a spring, preferably a helical spring. A spring is an elastic object that stores mechanical energy and releases it when the opposing force is removed.

The resilient bendable member is arranged longitudinally within the bendable portion such that the resilient bendable member extends through the bendable portion and supports the bendable portion.

The bendable portion may comprise an articulation arrangement comprising a plurality of adjacent articulation members aligned along the central longitudinal axis and arranged to articulate with respect to each other when the bending force is applied, wherein the resilient bendable member is arranged to align the articulation members along the central longitudinal axis and to support the articulation members.

The steering arrangement may comprise one or more articulation link members arranged along the articulation arrangement between the proximal end and the distal end. The articulation members comprise corresponding link channels and the one or more articulation link members extend through the corresponding link channels. The adjacent articulation members are linked to each other, preferably by respective joints or the one or more articulation link members.

The steering arrangement may comprise a control unit arranged at the side of the proximal end, wherein the one or more articulation link members are attached at their first ends to the control unit and at their opposite second ends to the distal end such that the control unit is adapted to pull respective one or more of the articulation link members to urge the bendable portion to bend such that the distal end is steered by the control unit.

The steering arrangement may comprise further an internal shaft extending through the external hollow shaft between the resilient bendable member and the control unit and engaging the resilient bendable member such that rotation of the internal shaft causes rotation of the resilient bendable member, wherein the control unit is adapted to rotate the internal shaft. The internal shaft is a hollow shaft.

The steerable articulating surgical instrument further may comprise an end effector arranged at the distal end, wherein the resilient bendable member is arranged to rotate the end effector, when the resilient bendable member rotates. The resilient bendable member engages the end effector such that rotation of the internal shaft causes rotation of the end effector about a longitudinal axis of the end effector.

The steering arrangement may comprise further an operation link member extending through the external hollow shaft between the end effector and the control unit and attached to the end effector. The control unit is adapted to pull the operation link member to apply tension and to operate the end effector.

The resilient bendable member defines a conduit along the bendable portion and the operation link member is arranged to extend through the conduit and the internal hollow shaft.

The control unit may comprise a handle having a first knob attached to the one or more articulation link members to pull the respective one or more articulation link members, a second knob engaging with the internal shaft to rotate the internal shaft and an operation trigger attached to the operation link member to operate the end effector. The control unit may comprise one or more locking switches to lock the first knob, the second knob and the operation trigger to fix the one or more articulation link members, the internal shaft and operation link member respectively.

The internal shaft may comprise screw gear and the second knob may engage the screw gear forming a screw conveyor such that a linear motion of the second knob would rotate the internal shaft about its longitudinal axis. Alternatively, the second knob may engage the internal shaft at a pivoting point such that a rotation of the second knob about the pivoting point would rotate the internal shaft about its longitudinal axis. The second knob may engage selectively via a switchable pin.

Other advantages of the present invention are defined in the appended claims.

### BRIEF DESCRIPTION OF THE DRAWINGS

The above described and other issues and features of the present invention will become more apparent from the following detailed description of preferred embodiments of the invention and the accompanying drawings.

FIG. 1a is a schematic illustration of a steerable articulating surgical instrument according to a first exemplary preferred embodiment of the present invention;

FIG. 1b shows a bendable portion of the steerable articulating surgical instrument of FIG. 1a;

FIG. 2 shows a steerable articulating surgical instrument according to a second exemplary preferred embodiment of the present invention;

FIG. 3 shows a knob portion of the steerable articulating surgical instrument of FIG. 2;

FIG. 4a shows an end effector and a distal end of the steerable articulating surgical instrument of FIG. 2;

FIG. 4b shows a bendable portion of the steerable articulating surgical instrument of FIG. 2;

FIG. 5 shows a operation trigger portion of the steerable articulating surgical instrument of FIG. 2;

FIG. 6 shows a steerable articulating surgical instrument according to a third exemplary preferred embodiment of the present invention;

FIG. 7 shows a knob portion of the steerable articulating surgical instrument of FIG. 6.

### DETAILED DESCRIPTION OF EXEMPLARY PREFERRED EMBODIMENTS

Preferred embodiments of the present invention are described below with reference to the accompanying drawings, in which the same reference numbers refer to the same features.

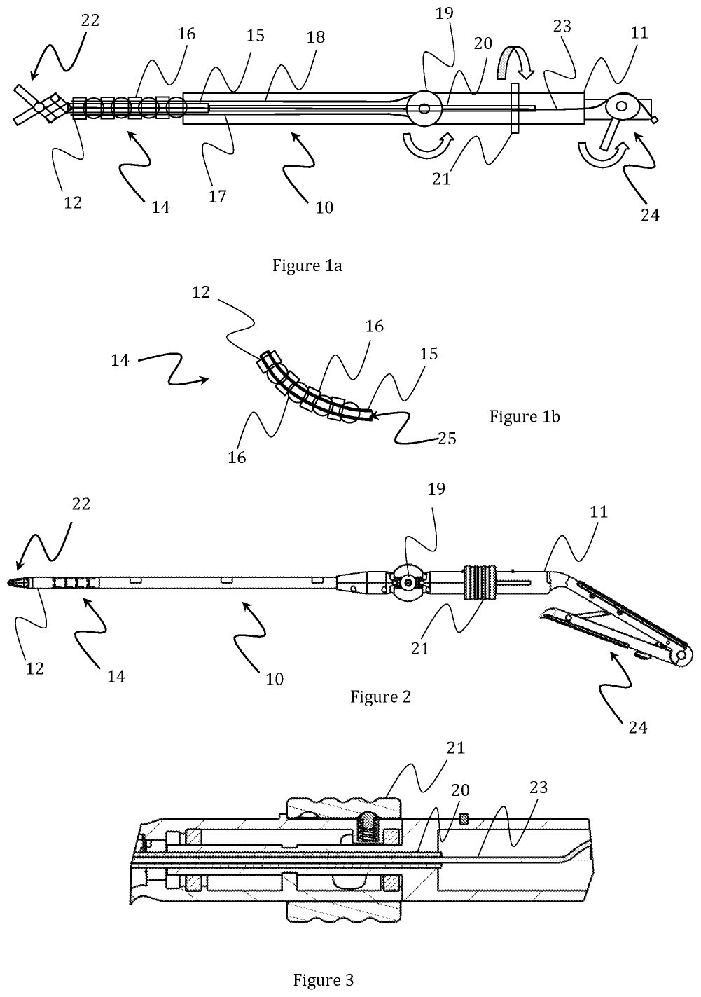

FIG. 1a shows a steerable articulating surgical instrument according to a first exemplary preferred embodiment of the present invention. The steerable articulating surgical instrument comprises an external hollow shaft 10 extending between a proximal end 11 and a distal end 12. The external hollow shaft 10 comprises a bendable portion 14 adapted to allow deflection of the distal end 12 to a deflected state as shown in FIG. 1b.

The steerable articulating surgical instrument comprises a steering arrangement adapted to apply a bending force to the bendable portion 14. The bendable portion 14 comprises a resilient bendable member 15 resisting the bending force. The resilient bendable member 15 is adapted to apply retraction of the deflected distal end 12 to an undeflected state.

The resilient bendable member 15 is adapted to store mechanical energy during bending of the bendable portion 14 when the bending force is applied and to release stored mechanical energy when the bending force is removed.

The external hollow shaft 10 extends along a central longitudinal axis, wherein the bending force is applied such that the bendable portion 14 is deflected from the central longitudinal axis to move radially away from the central longitudinal axis.

The steering arrangement is adapted to apply the bending force to urge the bendable portion 14 to a bent state as seen in FIG. 1b, wherein the distal end 12 is deflected.

The resilient bendable member 15 is adapted to apply retraction such that to align the bendable portion 14 along the central longitudinal axis and to support the bendable portion 14 to return to an unbent state as shown in FIG. 1a, wherein the distal end 12 is not deflected.

The resilient bendable member 15 is a helical spring made of spring steel. The resilient bendable member 15 may be an alternative resilient element made of an elastic and rigid material.

The resilient bendable member 15 is arranged longitudinally within the bendable portion 14 such that the resilient bendable member 15 extends through the bendable portion 14 and supports the bendable portion 14.

The bendable portion 14 comprises an articulation arrangement comprising a plurality of adjacent articulation members 16 aligned along the central longitudinal axis and arranged to articulate with respect to each other when the bending force is applied. The resilient bendable member 15 is arranged to align the articulation members 16 along the central longitudinal axis. The resilient bendable member 15 and the articulation members 16 support each other structurally.

The steering arrangement comprises two articulation link members 17, 18 arranged along the articulation arrangement between the proximal end 11 and the distal end 12. The articulation members 16 comprise corresponding link channels and the articulation link members 17, 18 extend through the corresponding link channels. The articulation link members 17, 18 link the adjacent articulation members 16 to each other. The articulation link members 17, 18 are made of cable or string.

The steering arrangement comprises a first knob 19 arranged at the side of the proximal end 11. The articulation link members 17, 18 are attached at their first ends to the first knob 19 and at their opposite second ends to the distal end 12. The first knob 19 may be rotated by an operator to pull respective one of the articulation link members 17, 18 to urge the bendable portion to bend the distal end to the side of the pulled articulation link member.

The steering arrangement comprises further an internal shaft 20 extending through the external hollow shaft 10 between the resilient bendable member 15 and a second knob 21. The second knob 21 engages the resilient bendable member 15 to rotate the internal shaft 20 and thereby rotate the resilient bendable member 15 and an end effector 22 attached thereto. The second knob 21 may be rotated by the operator to rotate the internal shaft 20. The internal shaft 20 is a hollow shaft.

The resilient bendable member 15 engages the end effector 22 such that rotation of the internal shaft 20 causes rotation of the end effector 22 about a longitudinal axis of the end effector 22.

The steering arrangement comprises further an operation link member 23 made of a cable or string extending through the external hollow shaft 10 between the end effector 22 and an operation trigger 24 and attached to the end effector 22. When the operation trigger 24 is triggered to pull the operation link member 23, tension is applied to the operation link member 23 and the end effector 22 is operated.

The resilient bendable member 15 defines a conduit 25 and the operation link member 23 is arranged to extend through the conduit 25 and the internal hollow shaft 20.

One or more locking switches are provided to lock the first knob 19, the second knob 21 and the operation trigger 24 to fix the articulation link members 17, 18 the internal shaft 20 and operation link member 23 respectively.

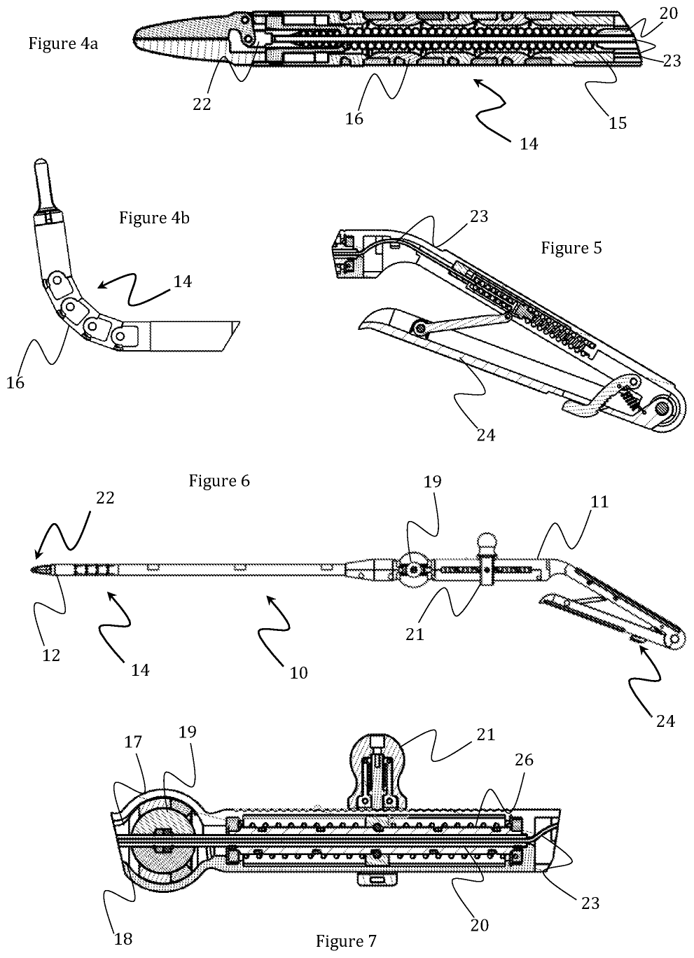

FIG. 2 shows a steerable articulating surgical instrument according to a second exemplary preferred embodiment of the present invention similar to the steerable articulating surgical instrument of FIG. 1a. The steerable articulating surgical instrument comprises an external hollow shaft 10 extending between a proximal end and a distal end 12. The external hollow shaft 10 comprises a bendable portion 14 adapted to allow deflection of the distal end 12 to a deflected state as shown in FIG. 4b.

The steerable articulating surgical instrument comprises a steering arrangement adapted to apply a bending force to the bendable portion 14. The bendable portion 14 comprises a resilient bendable member 15 resisting the bending force.

As shown in FIG. 4a, the resilient bendable member 15 is a helical spring made of spring steel. The adjacent articulation members 16 are linked to each other by respective joints as further shown in FIG. 4b and the one or more articulation link members.

The resilient bendable member 15 is arranged longitudinally within the bendable portion 14 such that the resilient bendable member 15 extends through the bendable portion 14 and supports the bendable portion 14.

The bendable portion 14 comprises an articulation arrangement comprising a plurality of adjacent articulation members 16 aligned along the central longitudinal axis and arranged to articulate with respect to each other when the bending force is applied.

The resilient bendable member 15 is arranged to align the articulation members 16 along the central longitudinal axis. The resilient bendable member 15 and the articulation members 16 support each other structurally.

The steering arrangement comprises two articulation link members 17, 18 arranged along the articulation arrangement between the proximal end 11 and the distal end 12. The articulation members 16 comprise corresponding link channels and the articulation link members 17, 18 extend through the corresponding link channels. The articulation link members 17, 18 link the adjacent articulation members 16 to each other. The articulation link members 17, 18 are made of cable or string.

The steering arrangement comprises a first knob 19 arranged at the side of the proximal end 11. The steering arrangement comprises further an operation link member 23 made of a cable or string extending through the external hollow shaft 10 between an end effector 22 (comprising a needle holder) and an operation trigger 24 and attached to the end effector 22. As shown in FIG. 5, when the operation trigger 24 is triggered to pull the operation link member 23, tension is applied to the operation link member 23 and the end effector 22 is operated.

The resilient bendable member 15 defines a conduit 25 and the operation link member 23 is arranged to extend through the conduit 25 and the internal hollow shaft 20 as shown in FIG. 4a.

FIG. 6 shows a steerable articulating surgical instrument according to a third exemplary preferred embodiment of the present invention similar to the steerable articulating surgical instrument of FIG. 2.

As shown in FIG. 7, the articulation link members 17, 18 are attached at their first ends to the first knob 19 and at their opposite second ends to the distal end 12. The first knob 19 may be rotated by an operator to pull respective one of the articulation link members 17, 18 to urge the bendable portion to bend the distal end to the side of the pulled articulation link member.

The steering arrangement comprises further an internal shaft 20 extending through the external hollow shaft 10 between the resilient bendable member 15 and the a second knob 21. The internal shaft 20 engages the resilient bendable member 15 to rotate the internal shaft 20 and thereby rotate the resilient bendable member 15 and an end effector 22 (a needle holder) attached thereto. The second knob 21 is rotated by the operator to rotate the internal shaft 20. The internal shaft 20 is a hollow shaft.

The internal shaft 20 comprises screw gear 26 and the second knob 21 engages the screw gear 26 forming a screw conveyor. A linear motion of the second knob 21 rotates the internal shaft 20 about its longitudinal axis.

The above description is not limiting. Many other examples or adaptations of the above embodiments will be apparent to those skilled in the art upon review of the above description without departing from the scope of the invention. Each of the following claims is incorporated herein as a separate preferred embodiment. Certain features of the disclosed embodiments are shown at an exaggerated scale or schematically, and some details of conventional elements are omitted for clarity and brevity.

The word “a” in this specification appropriately means “one or more” or “at least one”. The term “based on” can mean “using at least a part of . . . or a derivation of . . . ”. The word “first” can mean “initial”, “current” or “provisional” and vice versa. The word “subsequent” can mean “next” or “following” and vice versa. The word “previous” can mean “prior” and vice versa.

The preferred embodiments of the invention have been described in the drawings and the description of the preferred embodiments. While these disclosures directly describe the above embodiments, it is understood that those skilled in the art may consider modifications and/or variations to the specific embodiments shown and described herein.

1. A steerable articulating surgical instrument for surgery, comprising:
an external hollow shaft extending between a proximal end and a distal end, wherein the external hollow shaft comprises a bendable portion adapted to allow deflection of the distal end;
a steering arrangement adapted to apply a bending force to the bendable portion, wherein the bendable portion comprises a resilient bendable member adapted to resist the bending force.

2. The steerable articulating surgical instrument according to claim 1, wherein the resilient bendable member is adapted to store mechanical energy during bending of the bendable portion when the bending force is applied and to release stored mechanical energy to unbend the bendable portion when the bending force is removed.

3. The steerable articulating surgical instrument according to claim 1, wherein the external hollow shaft extends along a central longitudinal axis, wherein the bending force is applied such that the bendable portion is deflected from the central longitudinal axis to move radially away from the central longitudinal axis.

4. The steerable articulating surgical instrument according to claim 1, wherein the resilient bendable member comprises a spring.

5. The steerable articulating surgical instrument according to claim 1, wherein the resilient bendable member is arranged longitudinally within the bendable portion such that the resilient bendable member extends through the bendable portion and supports the bendable portion.

6. The steerable articulating surgical instrument according to claim 1, wherein the bendable portion comprises an articulation arrangement comprising a plurality of adjacent articulation members arranged to articulate with respect to each other when the bending force is applied, wherein the resilient bendable member is arranged to align the articulation members and to support the articulation members.

7. The steerable articulating surgical instrument according to claim 1, wherein the steering arrangement comprises one or more articulation link members arranged along the articulation arrangement and the external hollow shaft, the articulation members comprising corresponding link channels and the one or more articulation link members extending through the corresponding link channels.

8. The steerable articulating surgical instrument according to claim 1, wherein the steering arrangement comprises an internal shaft extending through the external hollow shaft engaging the resilient bendable member such that rotation of the internal shaft causes rotation of the resilient bendable member.

9. The steerable articulating surgical instrument according to claim 1, wherein the steerable articulating surgical instrument comprises an end effector, wherein the resilient bendable member is arranged to rotate the end effector, when the resilient bendable member rotates.

10. The steerable articulating surgical instrument according to claim 1, wherein the steering arrangement comprises an operation link member extending through the external hollow shaft and attached to the end effector, wherein the resilient bendable member defines a conduit along the bendable portion and the operation link member is arranged to extend through the conduit and the internal hollow shaft.

11. The steerable articulating surgical instrument according to claim 1, further comprising a control unit with a handle having a first knob attached to the one or more articulation link members to pull the respective one or more articulation link members, a second knob engaging with the internal shaft to rotate the internal shaft and a operation trigger attached to the operation link member to operate the end effector.
